(19) United States
(12) Patent Application Publication
(10) Pub. No.: US 2003/0083390 A1
(43) Pub. Date: May 1, 2003

(54) Fischer-tropsch tail-gas utilization

(76) Inventors: Lalit S. Shah, Sugar Land, TX (US); Pradeep S. Thacker, Houston, TX (US); Manual E. Quintana, Sugar Land, TX (US); Rae Song, Houston, TX (US)

Correspondence Address: STEPHEN H. CAGLE, HOWREY, SIMON, ARNOLD & WHITE, LLP, 750 BERING DRIVE, HOUSTON, TX, 77057, US

(21) Appl. No.: 10/001,820

(22) Filed: Oct. 23, 2001

(51) Int. Cl. C07C 27/00; C07C 27/06

(52) U.S. Cl. 518/702; 518/704

(57) ABSTRACT

The present invention deals with the handling of the tail-gas product from an integrated gasification reactor/Fischer-Tropsch reactor unit. The Fischer-Tropsch tail-gas is either recycled back for feed to the gasification unit to be converted into syngas, is processed in a CO₂ removal unit and recycled back to the feed of the Fischer-Tropsch reactor to improve the liquid product yield, or is sent to a power production unit for the generation of electric power.

### BACKGROUND OF THE INVENTION

The process and advantages of gasifying hydrocarbonaceous material into synthesis gas are generally known in the industry. In high temperature gasification processes, synthesis gas is commonly produced from gaseous combustible fuels, such as natural gas and/or associated gas, liquid organic fuels or combustible solid organic fuels, such as coal, residual petroleum, wood, tar sand, shale oil, and municipal, agriculture or industrial waste. The gaseous or liquid or solid combustible organic fuels are reacted with a reactive oxygen-containing gas, such as air, enriched air, or pure oxygen, and a temperature modifier, such as steam, in a gasification reactor to obtain the synthesis gas in a oxygen deficient environment.

In the reaction zone of a gasification reactor, the contents will commonly reach temperatures in the range of about 1,700° F. (930° C.) to about 3,000° F. (1650° C.), and more typically in the range of about 2,000° F. (1100° C.) to about 2,800° F. (1540° C.). Pressure will typically be in the range of about 1 atmosphere (100 KPa) to about 250 atmospheres (25,000 KPa), and more typically in the range of about 15 atmospheres (1500 Kpa) to about 150 atmospheres (1500 KPa).

In a typical gasification process, the synthesis gas will substantially comprise hydrogen (H₂), carbon monoxide (CO), and lessor quantities of impurities, such as water (H₂O), carbon dioxide (CO₂), carbonyl sulfide (COS) and hydrogen sulfide (H₂S). The synthesis gas is commonly treated to remove or significantly reduce the quantity of impurities, particularly H₂S, COS, and CO₂ before being utilized in downstream processes. A number of acid gas removal systems are commercially available and are known in the art. Selection of an appropriate acid gas removal system will usually depend on the degree of sulfur compounds and carbon dioxide removal required and by the operating pressure of the acid gas removal system. Determinations as to what type of acid gas system to use can easily be determined by one skilled in the art of acid gas removal from syngas.

It is well known in the art that synthesis gas, also commonly referred to as syngas, can be converted to hydrocarbons in the presence of a variety of transition metal catalysts. Such metals are commonly called Fischer-Tropsch catalysts, and are known to catalyze the conversion of CO and H₂ to hydrocarbons. Common catalysts are cobalt and iron on an alumina support. Other Group VIII metals such as ruthenium and osmium are also active. Other single metals that have been investigated as catalysts include rhenium, molybdenum, and chromium. The activities of these catalysts are commonly enhanced by the addition of a variety of metals, including copper, cerium, rhenium, manganese, platinum, iridium, rhodium, molybdenum, tungsten, ruthenium or zirconium, among others. The general chemistry of the much studied Fischer-Tropsch synthesis is as follows:

nCO+2nH₂→(—CH₂—)n+nH₂O+Heat (1)

CO+H₂O\vec{←}H₂+CO₂ (2)

The types and amounts of reaction products obtained via Fischer-Tropsch synthesis varies upon many conditions, such as reactor type, process conditions, and type of Fischer-Tropsch synthesis catalyst used. There are four main types of F-T reactors being used commercially: tubular fixed bed reactors, entrained bed reactors, fixed-fluidized bed reactors and slurry bubble column reactors. These reactors can operate in both high and low temperature Fischer Tropsch processes. There are generally two types of Fischer Tropsch synthesis catalysts, cobalt based and iron based catalysts. Typical products of the Fischer-Tropsch reaction include hydrocarbons from C₁ to C₂₀₀ or higher, with the bulk of the hydrocarbons product being in the C₁ to C₅₀ range with chain limiting catalyst. Most of the hydrocarbons produced are mixtures of olefins and paraffins. The Fischer-Tropsch reaction also produces varying amounts of carbon dioxide, water, and oxygenated components, including acids such as acetic acid, formic acid, propionic acid; alcohols such as methyl alcohol, ethyl alcohol, propyl alcohol, and longer chained alcohols; aldehydes, ketones and esters. Typically, these oxygenated components comprise 1 to 20 weight percent of the Fischer-Tropsch reaction product, and because of their water-soluble nature are commonly found in the wastewater product of a Fischer-Tropsch reactor. Some of the oxygenated compounds are also found in hydrocarbon phase. The amount of gaseous hydrocarbons, paraffin, olefins, CO₂, oxygenates, liquid hydrocarbons, water, etc. depends on the type of reactor, catalyst employed and process conditions. For example, iron catalysts generally produce longer chain hydrocarbons that are more olefinic, produce less amount of water, higher amounts of oxygenates and higher amounts of CO₂ as compared to cobalt catalyst. The Fischer-Tropsch reaction products are commonly divided into separate streams of tail-gas, liquid hydrocarbons, and wastewater.

The product from a Fischer-Tropsch reactor typically comprise water vapor, CO₂, N₂, unreacted syngas (H₂ and CO), gaseous hydrocarbons (C₁-C₅), liquid hydrocarbon (C₅+) products, and various oxygenates. Generally, most of the water vapor, liquid hydrocarbon products and oxygenates are condensed and separated. This leaves the desired liquid hydrocarbon product and the oxygenate containing wastewater. The liquid hydrocarbon is processed in downstream product upgrading section and waste water is usually sent to a water treatment step.

What remains is the tail-gas, which is comprised of water vapor, CO₂, CH₄, N₂, unreacted syngas (H₂ and CO), and vapor hydrocarbon products. The F-T tail gas can be recycled back to the gasification unit or can be recycled to the Fischer-Tropsch reactor inlet or burned as fuel.

Electric power can be generated efficiently in integrated gasification combined cycle (IGCC) systems. For IGCC applications, the synthesis gas is fired as fuel to a gas turbine system that drives a generator to produce electric power. Hot turbine exhaust can be passed to a heat recovery system to produce high pressure steam which can be expanded through a steam turbine to drive another electric generator to produce additional power. Such IGCC systems generate electricity in an efficient and environmentally sound manner.

The production of chemicals or liquid fuels from a portion of the synthesis gas, such as in a Fischer-Tropsch reactor, in a IGCC system is also well known and has the advantages of common operating facilities and economy of scale in the coproduction of electric power and chemicals. Several references in the background art describe existing technology for combined chemical plant/IGCC power plant operations.

### SUMMARY OF THE INVENTION

The present invention deals with the handling of the tail-gas product from a combined gasification and Fischer-Tropsch plant. There are three major alternatives for the tail-gas, the first being recycling the tail-gas as additional feed to the gasification unit. The second alternative is processing the tail-gas in a CO₂ removal unit and then recycling the tail-gas back to the feed of the Fischer-Tropsch reactor to improve the liquid product yield. Finally, the third alternative is to send the tail-gas to a power production unit for the generation of electric power.

### BRIEF DESCRIPTION OF THE DRAWINGS

FIG. 1 is a schematic diagram of one embodiment of the present invention.

### DESCRIPTION OF ILLUSTRATIVE EMBODIMENTS

.The feedstock for a gasification process is usually a hydrocarbonaceous material, that is, one or more materials, generally organic, which provide a source of hydrogen and carbon for the gasification reaction. In the present invention, carbonaceous fuel is obtained and prepared. for feeding to a gasification reactor. Carbonaceous fuel is any solid, liquid, and gaseous combustible organic material as single feed or combinations feed that can be used as feedstock to a gasification process for synthesis gas production.

The extent of feed preparation step will vary depending on the composition and physical nature of the feedstock. Generally, solid carbonaceous fuels will need to be liquefied with oil or water prior to feeding to the gasifier. Liquid and gaseous carbonaceous fuels may be suitable for direct feed to the gasifier, but can be pre-treated for removal of any impurities that might be present in the feed.

After the feed preparation step, the carbonaceous fuel is sent to a gasification reactor, or gasifier. In the gasifier, the carbonaceous fuel is reacted in an oxygen deficient environment with a reactive oxygen-containing gas, such as air or substantially pure oxygen having greater than about 90 mole percent oxygen, or oxygen enriched air having greater than about 21 mole percent oxygen. Substantially pure oxygen as produced in an air separation unit or produced by membrane technology is preferred. The partial oxidation of the hydrocarbonaceous material is completed, advantageously in the presence of a temperature control moderator such as steam, in a gasification zone to obtain hot synthesis gas.

In the gasification zone of a gasifier, the contents will commonly reach temperatures in the range of about 1,700° F. (927° C.) to 3,000° F. (1649° C.), and more typically in the range of about 2,000° F. (1093° C.) to 2,800° F. (1538° C.). Pressure will typically be in the range of about 1 atmospheres (101 kPa) to about 250 atmospheres (25331 kPa), and more typically in the range of about 15 atmospheres (1520 kPa) to about 150 atmospheres (15,199 kPa), and even more typically in the range of about 40 atmospheres (6080 kPa) to about 80 atmospheres (8106 kPa). See U.S. Pat. No. 3,945,942 describing a partial oxidation burner assembly. See U.S. Pat. No. 5,656,044 describing a method and an apparatus for the gasification of organic materials. See also U.S. Pat. Nos. 5,435,940, 4,851,013, and 4,159,238 describing a few of the many gasification processes known in the prior art. The entire disclosures of the above referenced patents are hereby incorporated by reference and relied upon.

The hot gasification process product, synthesis gas, or syngas, comprises carbon monoxide and hydrogen. Carbon Monoxide is a used as a major building block for many chemicals. Hydrogen is a commercially important reactant for hydrogenation reactions. Other materials often found in the synthesis gas include hydrogen sulfide, carbonyl sulfide, carbon dioxide, ammonia, cyanides, and particulates in the form of carbon and trace metals. The extent of the contaminants in the syngas is determined by the type of carbonaceous feed, the type of gasifier, and the gasifier operating conditions. In any event, the removal of these contaminants is critical to make gasification a viable process. Hydrogen sulfide, removal is particularly important.

As the product gas is discharged from the gasifier, it is usually subjected to a cooling and cleaning operation involving a scrubbing technique. The syngas from the gasifier is first introduced into a scrubber and contacted with a water spray which not only cools the gas but also removes particulate and ionic constituents from the synthesis gas. After removing the particulates and cooling the syngas, the cooled gas is then treated to desulfurize the gas prior to utilization of the synthesis gas.

The synthesis gas acid gas removal facilities using either amine or physical solvents, removes the acid gases, particularly hydrogen sulfide. The acid gas removal facilities typically operate at lower temperatures. After the synthesis gas is cooled to below about 130° C., preferably below about 90° C., the contaminants in the gas, especially sulfur compounds and acid gases, can be readily removed. The synthesis gas is contacted with the solvent in an acid gas removal contactor. Said contactor may be of any type known to the art, including trays or a packed column. Operation of such an acid removal contactor is well known in the art. The cleaned syngas can be used for many downstream processing. The degree of acid gas removal varies with the downstream use of syngas. The recovered acid gases are send to various recovery processes.

After being processed in the acid gas removal step, the syngas is sent to a hydrocarbon synthesis reactor, such as a Fischer-Tropsch reactor, where it is contacted with a hydrocarbon synthesis catalyst. Hydrocarbon synthesis catalyst converts synthesis gas into hydrocarbon products. Common catalysts are cobalt and iron on an alumina support. Other Group VIII metals such as ruthenium and osmium are also active. Other single metals that have been investigated as catalysts include rhenium, molybdenum, and chromium. The activities of these catalysts are commonly enhanced by the addition of a variety of metals, including copper, cerium,. rhenium, manganese, platinum, iridium, rhodium, molybdenum, tungsten, ruthenium or zirconium. Many other metals can be used, and it is within the scope of this invention to include all catalysts that convert synthesis gas in to hydrocarbon products. See U.S. Pat. Nos. 5,780,391, 5,162,284, 5,102,581, 4,801,573, and 4,686,238 for illustrations of some of the various types of catalyst that can be used to produce hydrocarbons from synthesis gas. The entire disclosures of the above referenced patents are hereby incorporated by reference and relied upon. The wide range of catalysts and catalyst modifications disclosed in the art directly correspond to an equally wide range of conversion conditions in the hydrocarbon synthesis reactor. Catalyst selection can provide some flexibility toward obtaining selected types of products, and some control over their molecular weight distribution.

The types and amounts of reaction products obtained via Fischer-Tropsch synthesis varies upon many conditions, such as reactor type, process conditions, and type of Fischer-Tropsch synthesis catalyst used. There are four main types of F-T reactors being used commercially: tubular fixed bed reactors, entrained bed reactors, fixed-fluidized bed reactors and slurry bubble column reactors. These reactors can operate in both high and low temperature Fischer Tropsch processes. There are generally two types of Fischer Tropsch synthesis catalysts, cobalt based and iron based catalysts. Typical products of the Fischer-Tropsch reaction include hydrocarbons from C₁ to C₂₀₀ or higher, with the bulk of the hydrocarbons product being in the C₁ to C₅₀ range with chain limiting catalyst. Most of the hydrocarbons produced are mixtures of olefins and paraffins. The Fischer-Tropsch reaction also produces varying amounts of carbon dioxide, water, and oxygenated components, including acids such as acetic acid, formic acid, propionic acid; alcohols such as methyl alcohol, ethyl alcohol, propyl alcohol, and longer chained alcohols; aldehydes, ketones and esters. Typically, these oxygenated components comprise 1 to 20 weight percent of the Fischer-Tropsch reaction product, and because of their water-soluble nature are commonly found in the wastewater product of a Fischer-Tropsch reactor. Some of the oxygenated compounds are also found in hydrocarbon phase. The amount of gaseous hydrocarbons, paraffin, olefins, CO₂, oxygenates, liquid hydrocarbons, water, etc. depends on the type of reactor, catalyst employed and process conditions. For example, iron catalysts generally produce longer chain hydrocarbons that are more olefinic, produce less amount of water, higher amounts of oxygenates and higher amounts of CO₂ as compared to cobalt catalyst. The Fischer-Tropsch reaction products are commonly divided into separate streams of tail-gas, liquid hydrocarbons, and wastewater.

The Fischer-Tropsch liquid hydrocarbon stream (light and heavy) is the desired product of the hydrocarbon synthesis reactor system. This stream comprises any condensed hydrocarbons that have been separated from the condensed wastewater stream or removed directly from the reactor. This stream typically includes hydrocarbons chains from C₅ to C₂₀₀ or higher.

The Fischer-Tropsch liquid wastewater stream is the water product of the hydrocarbon synthesis reactor system that has been condensed and separated from the light Fischer-Tropsch liquids. This wastewater stream is usually comprised of water and the water soluble oxgynated components such as acids, alcohols, aldehydes, ketones and esters. Small amounts of hydrocarbons can also be found in the wastewater stream, subject to their solubility at the temperatures and pressures at which the condensation takes place. This wastewater stream is normally passed to a water treatment facility where it undergoes typical water treatment steps known in the art, such as anaerobic digestion and biological oxidation, in order to remove the contaminants and produce clean water for disposal or use.

The Fischer-Tropsch tail-gas stream is the gaseous product of a Fischer-Tropsch reactor that does not condense when the reaction products are cooled. The tail-gas is typically comprised of unconverted syngas and uncondensed products, typically CO, H₂, CO₂, gaseous hydrocarbons (C₁-C₅), H₂O, N₂, Ar, and, depending on the catalyst, other compounds and hydrocarbons.

Water is known to be a powerful inhibitor in the Fischer-Tropsch synthesis. Carbon dioxide is also an inhibitor, but very much weaker than water. This is why it is desirable to remove CO₂ from the syngas prior to processing in a Fischer-Tropsch reactor. Water is generally produced by the primary step in the conversion process from equation (1) above, but for iron catalyst much of the water is consumed by the reversible water gas shift reaction from equation (2) above. For cobalt catalyst the reverse water gas shift is not predominant. Thus, regardless of whether the selected hydrocarbon synthesis catalyst produces primarily H₂O, from equation (1), or CO₂, from equation (2), CO2 is usually a significant component of the tail-gas. Generally, depending on the conversion of the syngas obtained in the Fischer Tropsch synthesis, the tail gas also contains large amounts of unconverted syngas. Typically, the tail-gas is recycled back to the syngas feed stream to the Fischer-Tropsch reactor to improve the liquid product yield.

Depending on the Fischer-Tropsch catalyst employed, the tail-gas may also contain varying amounts of hydrocarbons. Recycling the tail-gas back to the gasifier can then convert these hydrocarbons into syngas, thus producing another step to increase the overall yield of the desired Fischer-Tropsch liquid hydrocarbon product.

Recycling the tail-gas to the gasifier and/or to the Fischer-Tropsch reactor increases the conversion to Fischer-Tropsch liquid hydrocarbons. However, the cost of the related and downstream equipment also increases. Therefore, a third alternative for the tail-gas, namely power generation, may also provide an economic alternative to recycling the tail-gas back into the integrated gasification/Fischer-Tropsch process. The tail-gas is combusted and the combusted gas is used to produce power directly by expanding the combusted gas through a gas turbine, or indirectly by generation of steam and expansion of that steam through a turbine. Commonly, the gasifier syngas product is used in this manner for power production. It is possible that if a fraction of syngas is used for power generation and fraction used for Fischer Tropsch synthesis, the syngas and the tail-gas could be combined with it prior to combustion in the gas turbine. For gas turbine, the BTU value and the composition of the feedgas are key parameters for determining if sending the tail-gas to a gas turbine is a viable alternative. This altrenative is also influenced by the amount of tail-gas that is recycled to the gasifier and/or the Fischer-Tropsch reactor.

Thus, the tail-gas could be individually recycled to the gasifier or the Fischer-Tropsch reactor or sent to the gas turbine. Otherwise, the tail-gas could be sent in some combination, and the flow split to two alternatives, or to all three alternatives. The specifics of each project such as the catalyst used, feedstock used, the price of power, the price and desired composition of the Fischer-Tropsch liquids, and the price of the carbonaceous feedstock are all items to be considered in determining the optimum arrangement for tail-gas utilization.

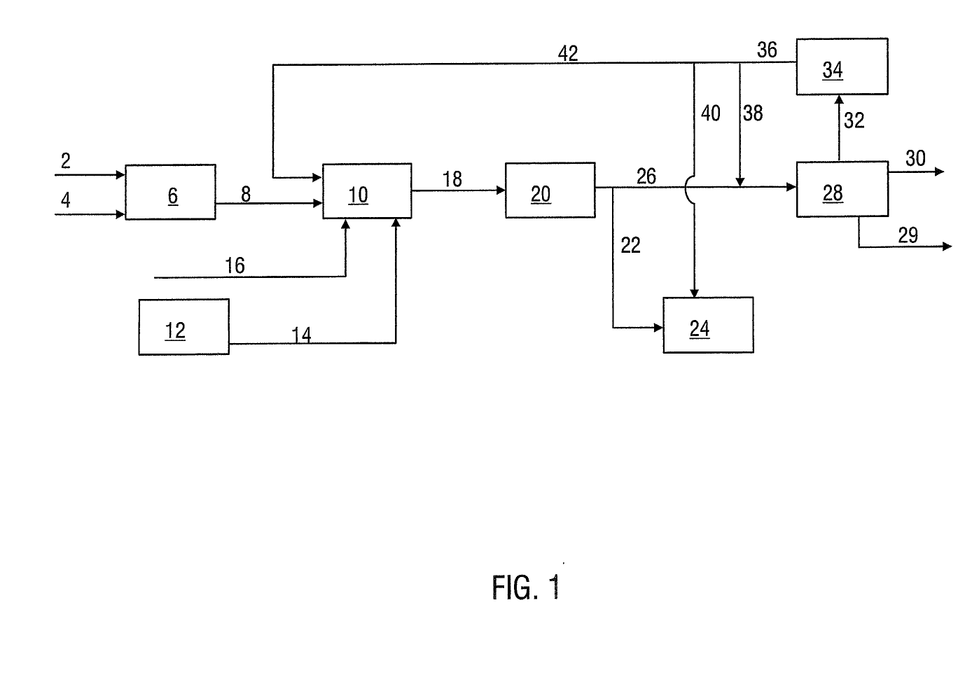

Referring now to FIG. 1, solid carbonaceous fuel 2 and water 4 are sent to a slurry preparation step 6 to produce liquefied solid carbonaceous feedstock 8. For the gaseous and liquid feed slurry preparation step 6 will not be necessary and feedstock 8 will be the gaseous or liquid feed. The feedstock 8 is then sent to gasifier 10, along with oxygen 14, usually from an air separation unit 12, and steam 16, used as a temperature moderator. At least a portion 42 of the tail-gas product 32 or the entire tail gas stream 32 from the downstream Fischer-Tropsch reactor 28 is also sent to the gasifier 10. The gasifier 10 syngas product 18 is then sent to acid gas unit 20, where a substantial portion of the impurties of the syngas 18 are removed. A portion of the sweetened syngas 22 can then be sent to power block 24, where it is likely to be combusted and expanded across a turbine to generate power, and/or is used to produce steam that can also be used to generate power. It is possible that all of the syngas from the acid gas removal unit 20 is sent to the Fischer Tropsch unit. Hence either some or all of the portion of the sweetened syngas 26 is sent to Fischer-Tropsch reactor 28, where it is reacted with a catalyst to from wastewater 29, liquid synthetic hydrocarbons 30, and tail-gas 32. There are two alternates with tail gas 32. One alternate is to process tail gas through the second acid gas removal unit to remove CO₂. The second alternate is to send tail-gas as it Is without CO₂ removal. In the first alternate the tail-gas 32 is processed in a second acid gas unit 34, where a substantial portion of the CO₂ present in the tail-gas 32 is removed. The sweetened tail-gas 36 can then be divided among three options: 1) recycled 38 back to the Fischer-Tropsch reactor 28 for additional hydrocarbon synthesis; 2) sent to the power block 24 for additional power generation; and at least 3) recycled back to the gasifier 10 for additional syngas production. In the second alternate the tail-gas 32 can be divided among two options: 1) recycled 46 directly to the gasifier 10 for additional syngas production; and 2) recycled 44 to the power block 24 for additional power generation. Each of the above options are viable as whole or in combinations with other.

While the devices, compositions and methods of this invention have been described in terms of preferred embodiments, it will be apparent to those of skill in the art that variations may be applied to the process described herein without departing from the concept and scope of the invention. All such similar substitutes and modifications apparent to those skilled in the art are deemed to be within the scope and concept of the invention as it is set out in the following claims.

What is claimed is:

1. A hydrocarbon synthesis process comprising:
(a) forming a synthesis gas by reacting a combustible carbonaceous material and a tail-gas with 1) steam and/or water and 2) oxygen or air or enriched air at an elevated temperature in a gasification reactor;
(b) contacting the said synthesis gas with a hydrocarbon synthesis catalyst to form liquid hydrocarbons and the tail-gas in hydrocarbon synthesis reactor;
(c) separating the resulting tail-gas and the liquid hydrocarbons; and
(d) recycling the tail-gas back the reactor.

2. The process of claim 1 comprising the additional step of removing carbon dioxide from a fraction of the tail-gas and mixing the carbon dioxide-free tail-gas fraction with the synthesis gas prior to contacting the synthesis gas with the hydrocarbon synthesis catalyst.

3. The process of claim 1 comprising the additional step of combusting a fraction of the tail-gas and generating power from said combusted fraction.

4. The process of claim 3 comprising the additional step of removing carbon dioxide from a second fraction of the tail-gas and mixing the carbon dioxide-free tail-gas second fraction with the synthesis gas prior to contacting the synthesis gas with the hydrocarbon synthesis catalyst.

5. A method for consuming a tail-gas produced by reacting a synthesis gas with a hydrocarbon synthesis catalyst comprising reacting the tail-gas and a combustible carbonaceous material with steam and oxygen at an elevated temperature to form the synthesis gas.

6. The method of claim 5 comprising the additional step of removing carbon dioxide from a fraction of the tail-gas and mixing the carbon dioxide-free tail-gas fraction with the synthesis gas prior to reacting the synthesis gas with the hydrocarbon synthesis catalyst.

7. The method of claim 5 comprising the additional step of combusting a fraction of the tail-gas and generating power from said combusted fraction.

8. The method of claim 7 comprising the additional step of removing carbon dioxide from a second fraction of the tail-gas and mixing the carbon dioxide-free tail-gas second fraction with the synthesis gas prior to reacting the synthesis gas with the hydrocarbon synthesis catalyst.
